(19) United States
(12) Patent Application Publication
(10) Pub. No.: US 2017/0156645 A1
(43) Pub. Date: Jun. 8, 2017

(54) NON-INVASIVE BLOOD GLUCOSE MEASURING DEVICE AND MEASURING METHOD USING THE SAME

(71) Applicant: INDUSTRIAL TECHNOLOGY RESEARCH INSTITUTE, Hsinchu (TW)

(72) Inventors: Hong-Dun Lin, Hsinchu City (TW); Yen-Hsien Lee, Taipei City (TW)

(73) Assignee: INDUSTRIAL TECHNOLOGY RESEARCH INSTITUTE, Hsinchu (TW)

(21) Appl. No.: 14/980,416

(22) Filed: Dec. 28, 2015

(30) Foreign Application Priority Data
Dec. 3, 2015 (TW) 104140564

(51) Int. Cl. A61B 5/145; A61B 5/00

(52) U.S. Cl. CPC A61B 5/14532; A61B 5/7225; A61B 5/6826; A61B 5/7275

(57) ABSTRACT

A non-invasive blood glucose measuring device and a measuring method using the same are provided. The non-invasive blood glucose measuring device includes a signal emitting module, a delay component, a wave mixing component and a signal processor. The signal emitting module is configured to emit a detection signal. The delay component is configured to delay the detection signal to become a delay signal. The wave mixing component is configured to mix a return signal and the delay signal to form a mixed signal, wherein the return signal is the detection signal reflected from an object. The signal processor is configured to obtain a blood glucose level of the object according to the mixed signal.

This application claims the benefit of Taiwan application Serial No. 104140564, filed Dec. 3, 2015, the disclosure of which is incorporated by reference herein in its entirety.

### TECHNICAL FIELD

The disclosure relates in general to a non-invasive blood glucose measuring device and a measuring method using the same, and more particularly to a non-invasive blood glucose measuring device using a delay signal and a measuring method using the same.

### BACKGROUND

Along with the improvement in living quality, people's diet is getting richer and richer, but at the same time more and more people have developed diabetes. In response to the needs of the diabetics, a blood glucose measuring device for residential use is provided. Conventional blood glucose measuring device normally pricks the user's finger to take blood and then analyzes blood glucose of the sampled blood drop. However, the users do not feel comfortable with such invasive blood glucose measuring device, and the pricked finger is susceptible to infection.

Therefore, it has become a prominent task for the industries to provide a new blood glucose measuring device to resolve the above problems.

### SUMMARY

According to one embodiment, a non-invasive blood glucose measuring device is provided. The non-invasive blood glucose measuring device includes a signal emitting module, a delay component, a wave mixing component and a signal processor. The signal emitting module is configured to emit a detection signal. The delay component is configured to delay the detection signal to become a delay signal. The wave mixing component is configured to mix a return signal and the delay signal to form a mixed signal, wherein the return signal is the detection signal reflected from an object. The signal processor is configured to obtain a blood glucose level of the object according to the mixed signal.

According to another embodiment, a non-invasive blood glucose measuring method is provided. The non-invasive blood glucose measuring method includes following steps: emitting a detection signal; delaying the detection signal to become a delay signal; mixing a return signal and the delay signal to form a mixed signal, wherein the detection signal is the detection signal reflected from an object; obtaining a blood glucose level of the object according to the mixed signal.

The above and other aspects of the disclosure will become better understood with regard to the following detailed description of the preferred but non-limiting embodiment (s). The following description is made with reference to the accompanying drawings.

### BRIEF DESCRIPTION OF THE DRAWINGS

FIG. 1 is a flowchart of a non-invasive blood glucose measuring method according to an embodiment of the present disclosure;

FIG. 2 is a functional block diagram of a non-invasive blood glucose measuring device according to an embodiment of the present disclosure; and

FIG. 3 is a diagram of a relationship between a blood glucose concentration and a signal intensity according to an embodiment of the present disclosure.

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

### DETAILED DESCRIPTION

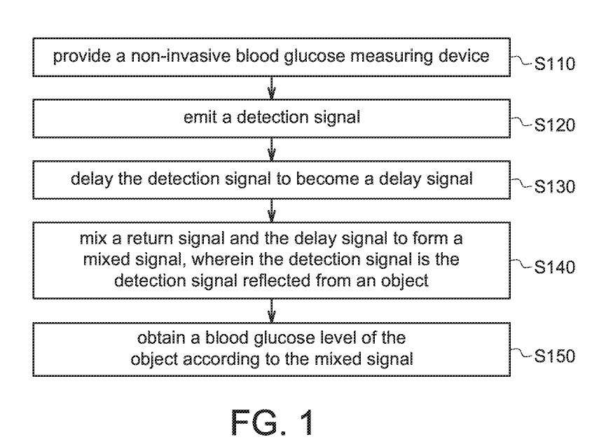

Referring to FIG. 1, a flowchart of a non-invasive blood glucose measuring method according to an embodiment of the present disclosure is shown.

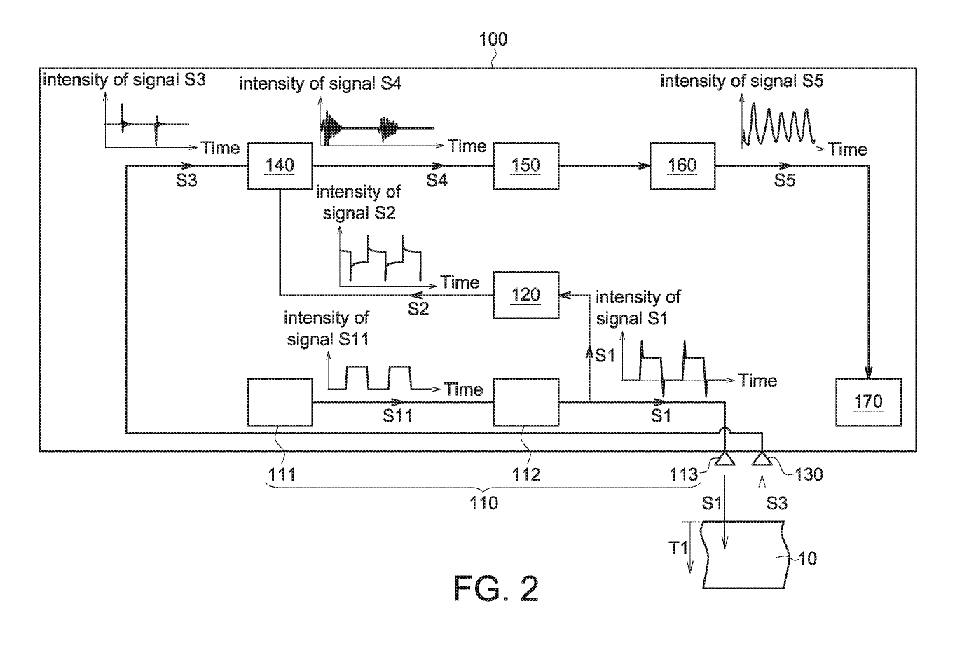

In step S110, a non-invasive blood glucose measuring device 100 as indicated in FIG. 2 is provided. FIG. 2 is a functional block diagram of a non-invasive blood glucose measuring device 100 according to an embodiment of the present disclosure.

The non-invasive blood glucose measuring device 100 can measure a blood glucose level of an object 10. The object 10 is such as a biological body's finger or other parts, and the biological body can be a human or an animal. In an embodiment, the non-invasive blood glucose measuring device 100 can be can be realized by a portable device, such as a wearable device that can be worn on the biological body's finger, wrist or other parts. Or, the non-invasive blood glucose measuring device 100 can be realized by a fixed device that can be fixed at a particular place such as hospital, shop or residential environment. Additionally, the non-invasive blood glucose measuring device 100 can measure the biological body's blood glucose level through direct contact or by keeping a distance from the biological body. That is, the non-invasive blood glucose measuring device 100 can be a contact type or a non-contact type blood glucose measuring device.

The non-invasive blood glucose measuring device 100 includes a signal emitting module 110, a delay component 120, a receiving antenna 130, a wave mixing component 140, a differential amplifier 150, a low-pass filter 160 and a signal processor 170.

The signal emitting module 110 can emit a detection signal S1 to the object 10. The delay component 120 can delay the detection signal S1 to become a delay signal S2. The wave mixing component 140 can mix a return signal S3 and the delay signal S1 to form a mixed signal S4, wherein the return signal S3 is the detection signal S1 reflected from the object 10. The signal processor can obtain a blood glucose level of the object 10 according to the mixed signal S4. Detailed descriptions of the non-invasive blood glucose measuring device 100 are disclosed below.

In step S120, a detection signal S1 is emitted to the object 10 by the signal emitting module 110. In an embodiment, the signal emitting module 110 is a radio frequency (RF) emitter, and the detection signal S1 emitted by the signal emitting module 110 is an RF signal, such as a nanosecond short pulse near-field sensing signal with a frequency of 300 MHz.

The signal emitting module 110 includes a pulse width modulator 111, an overshot/undershot band generator 112 and a transmitting antenna 113. The pulse width modulator 111 outputs a modulation signal S11. The modulation signal S11 is processed by the overshot/undershot band generator 112 to become the detection signal S1 with an overshot band and an undershot band. In an embodiment, the overshot/undershot band generator 112 can be omitted, and under such design, the detection signal S1 does not have an overshot band or an undershot band. After the detection signal S1 is generated, the detection signal S1 can be transmitted to the object 10 by a transmitting antenna 113. In an embodiment, the transmitting antenna 113 can be realized by such as a comb antenna, but the present disclosure is not limited thereto.

In step S130, delay the detection signal S1 can be delayed by the delay component 120 to become a delay signal S2. For example, the detection signal S1 transmitted to the object 10 can be concurrently transmitted to the delay component 120. The delay component 120 can delay the detection signal S1 to become a delay signal S2, which is lagged behind the detection signal S1 by a phase difference. That the delay signal S2 is lagged behind the detection signal S1 by a phase difference implies that the non-invasive blood glucose measuring device 100 receives a return signal S3 in a direction away from the object 10, and can receive a return wave reflected from a thicker portion T1 of the object 10. Thus, without changing the distance between the non-invasive blood glucose measuring device 100 and the object 10, a return wave can be received from the thicker portion T1 of the object 10. The thicker portion T1 can be a partial or total thickness of the object 10.

As indicated in FIG. 2, since the detection signal S1 decays during the transmission process, the amplitude of the delay signal S2 is smaller than that of the detection signal S1, and the pulse width of the delay signal S2 is narrower than that of the detection signal S1.

In step S140, a return signal S3 and the delay signal S2 are mixed to form a mixed signal S4 by the wave mixing component 140, wherein the return signal S3 is the detection signal S1 reflected from the object 10. For example, after the return signal S3 is received by the receiving antenna 130, the return signal S3 is transmitted to the wave mixing component 140. In an embodiment, the receiving antenna 130 can be realized by an asymmetric receiving antenna. The wave mixing component 140 mixes the return signal S3 and the delay signal S2 to form a mixed signal S4.

Then, the mixed signal S4 is sequentially processed by the differential amplifier 150 and the low-pass filter 160 to filter off high-frequency noises and become a mixed signal S5.

In step S150, a blood glucose level of the object 10 can be obtained by the signal processor 170 according to the mixed signal S5. For example, the signal processor 170 can firstly perform digital sampling on the mixed signal S5 to obtain a single intensity level corresponding to the mixed signal S5 and then obtain the blood glucose level corresponding to the mixed signal S5 according to an intensity of the mixed signal S5 and a relationship R1 between a blood glucose concentration and a signal intensity. In an embodiment, the signal processor 170 can be realized by such as a micro controller unit (MCU). Besides, the signal processor 170 can include a digital to analog converter (ADC) for performing the said digital sampling on the mixed signal S5.

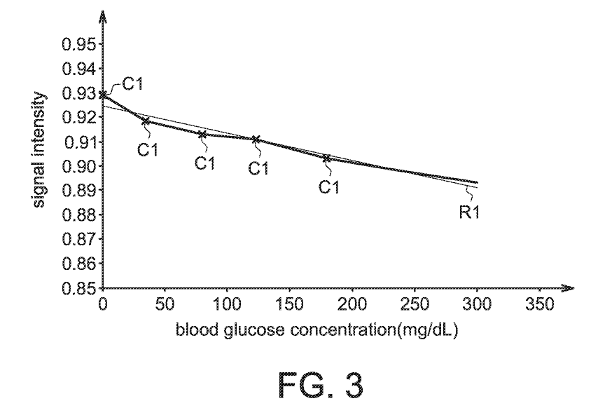

Referring to FIG. 3, a diagram of the relationship R1 between the blood glucose concentration and the signal intensity according to an embodiment of the present disclosure is shown. The relationship R1 is based on the results of experiments. In FIG. 3, the horizontal axis represents blood glucose concentration and the vertical axis represents signal intensity. The levels of signal intensities are normalized to be between 0˜1. The relationship R1 can be stored in the signal processor 170 or other storage unit such as memory.

The experiment points C1 of FIG. 3 represent different blood glucose concentrations of the same biological body. It can be known from the distribution of the experiment points C1 that different blood glucose concentrations correspond to different intensity levels. A linear equation corresponding to a number of experiment points C1 can be obtained by using the linear model such as the least square method. That is, the relationship R1 can be a linear curve. In an embodiment, the relationship R1 can be a fitted curve.

The signal processor 170 can calculate the blood glucose concentration corresponding to the intensity level of the mixed signal S5 according to the relationship R1 to obtain the corresponding blood glucose level. The non-invasive blood glucose measuring device 100 of the present embodiment can continuously detect the user's blood glucose and calculate the variation in blood glucose.

According to the embodiments of the present disclosure, the variation in human body's blood glucose is detected by a low-frequency (such as 300 MHz) nanosecond short pulse near-field sensing signal according to the nanosecond short pulse near-field radar sensing technique. Information of human body's blood glucose can be obtained from the radio frequency (RF) signal and the decay analysis of the receiving signal.

It will be apparent to those skilled in the art that various modifications and variations can be made to the disclosed embodiments. It is intended that the specification and examples be considered as exemplary only, with a true scope of the disclosure being indicated by the following claims and their equivalents.

What is claimed is:

1. A non-invasive blood glucose measuring device, comprising:
a signal emitting module configured to emit a detection signal;
a delay component configured to delay the detection signal to become a delay signal;
a wave mixing component configured to mix a return signal and the delay signal to form a mixed signal, wherein the return signal is the detection signal reflected from an object; and
a signal processor configured to obtain a blood glucose level of the object according to the mixed signal.

2. The non-invasive blood glucose measuring device according to claim 1, wherein the signal emitting module comprising:
a pulse width modulator configured to output a modulation signal;
an overshot/undershot band generator configured to process the modulation signal to become the detection signal having an overshot band and an undershot band; and
a transmitting antenna configured to transmit the detection signal to the object.

3. The non-invasive blood glucose measuring device according to claim 1, further comprising:
a receiving antenna configured to receive the return signal;
a differential amplifier; and
a low-pass filter;
wherein the mixed signal is sequentially processed by the differential amplifier and the low-pass filter.

4. The non-invasive blood glucose measuring device according to claim 1, further comprising a relationship between a blood glucose concentration and a signal intensity, wherein the signal processor is configured to obtain the blood glucose level corresponding to the mixed signal according to an intensity of the mixed signal and the relationship.

5. The non-invasive blood glucose measuring device according to claim 4, wherein the relationship is a linear equation.

6. The non-invasive blood glucose measuring device according to claim 1, wherein the detection signal is a radio frequency (RF) signal.

7. The non-invasive blood glucose measuring device according to claim 1, the detection signal is a nanosecond short pulse near-field sensing signal with a frequency of 300 MHz.

8. A non-invasive blood glucose measuring method, comprising:
emitting a detection signal;
delaying the detection signal to become a delay signal;
mixing a return signal and the delay signal to form a mixed signal, wherein the detection signal is the detection signal reflected from an object; and
obtaining a blood glucose level of the object according to the mixed signal.

9. The non-invasive blood glucose measuring method according to claim 8, further comprising:
outputting a modulation signal;
processing the modulation signal to become the detection signal having an overshot band and an undershot band; and
transmitting the detection signal to the object.

10. The non-invasive blood glucose measuring method according to claim 8, further comprising:
a receiving antenna configured to receive the return signal;
a differential amplifier; and
a low-pass filter;
wherein the mixed signal is sequentially processed by the differential amplifier and the low-pass filter.

11. The non-invasive blood glucose measuring method according to claim 8, further comprising:
obtaining the blood glucose level corresponding to the mixed signal according to an intensity of the mixed signal and a relationship between a blood glucose concentration and a signal intensity.

12. The non-invasive blood glucose measuring method according to claim 11, wherein the relationship is a linear equation.

13. The non-invasive blood glucose measuring method according to claim 12, wherein the detection signal is a radio frequency (RF) signal.

14. The non-invasive blood glucose measuring method according to claim 8, wherein the detection signal is a nanosecond short pulse near-field sensing signal with a frequency of 300 MHz.
